(19) United States
(12) Patent Application Publication
(10) Pub. No.: US 2007/0137806 A1
(43) Pub. Date: Jun. 21, 2007

(54) Rapid sampling assembly for thermo-mechanical pulp control application

(75) Inventor: Manpreet S. Sidhu, North Vancouver, CA (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International Inc.

(21) Appl. No.: 11/304,250

(22) Filed: Dec. 15, 2005

(51) Int. Cl. D21C 7/14; G01N 33/34

(52) U.S. Cl. 162/49; 73/53.03

(57) ABSTRACT

A technique for latency removal which can be effected without introducing a significant lag time can advance analysis of pulp for feedback control thermo-mechanical pulp applications. The process for analyzing a pulp material includes: (a) collecting a representative sample of the pulp material, (b) adjusting the consistency of the sample to yield an adjusted pulp sample, (c) employing a disintegrator to release latency from the pulp that is in the adjusted pulp sample to form a pulp composition that is substantially free of latent properties, and (d) analyzing the pulp composition to measure at least one pulp quality.

### FIELD OF THE INVENTION

This invention relates to pulp refining and papermaking and particularly to techniques for controlling the production and the quality of the pulp used in a papermaking process by employing a technique that efficiently and quickly removes latency from thermo-mechanical pulp (TMP) so that analysis of the pulp can be accomplished with minimum lag time to enable faster control of the TMP process.

### BACKGROUND OF THE INVENTION

Processes for making paper pulp consist in reducing the raw materials to separate fibers containing a greater or lesser amount of cellulose depending on the qualities which the pulp produced is required to have. The processes essentially consist of grinding operations, which are basically mechanical, which may be combined with more or less powerful delignification operations, which are basically chemical.

Depending on the relative importance of the two treatments, it is possible to distinguish five major types of pulp:
(1) Mechanical pulp, obtained by grinding without any chemical treatment beforehand of the raw material;
(2) Thermo-mechanical pulp, obtained by grinding under pressure, which is made easier by steaming the raw material beforehand to soften the lignin;
(3) Mechano-chemical pulp, obtained by grinding in combination with in situ or ex situ preliminary treatment of the raw material with chemical reagents;
(4) Semi-chemical pulp, obtained by grinding raw material which is previously subjected to partial chemical “cooking” under pressure; and
(5) Chemical pulp, where the chemical processing is much more powerful and produces both the delignification and the major part of the reduction to fiber.

Refiner mechanical pulp (RMP) is produced by the mechanical reduction of wood chips (and sometimes sawdust) in a disc refiner. The process usually involves the use of two refining stages operating in series, i.e., two-stage refiring, and produces a longer-fibered pulp than conventional ground wood. As a result, it is stronger, freer, bulkier, but usually somewhat darker in color, than that of stone ground wood. Thermo-mechanical pulping (TMP) was the first major modification of RMP, and is still employed on a large scale to produce high-tear pulps for newsprint and board. This process involves steaming the raw material under pressure for a short period of time prior to and during refining. The steaming serves to soften the chips, with the result that the pulp produced has a greater percentage of long fibers and fewer shives than RMP. The refined pulp from the TMP process is stored in a latency chest that allows the beaten fibers to relax in hot water to remove latency. In the latency chest, pulp is agitated at a consistency of about 1.25% in a temperature range generally between about 70° C. to 90° C. for 20 to 30 minutes or more. A fiber quality monitor that collects samples and determines pulp quality parameters such as freeness and fiber length distribution, as measured by various standards, is typically positioned at the exit of the latency chest.

It is becoming increasingly important to produce TMP pulp that is both uniform and of a high quality. Papermakers desire to optimize paper machine operations, and in some instances to replace the expensive kraft furnish. Even though advanced process control has gained general acceptance in the pulp and paper industry, the therno-mechanical pulping process is still under manual control in most pulp mills. Reliance on manual control stems primarily from the complexity of the TMP process, which is highly interactive requiring control and variable inputs from many sections of the refining process. Additionally, control of the TMP process is further complicated as blow-line consistency in most cases is not measured using an online sensor. Pulp quality descriptive variables such as fiber length and freeness are also measured infrequently as operation of the latency chest requires a residence time of at least 30 minutes to ensure complete removal of the latency.

In order to produce high quality thermo-mechanical pulp, the refining process must be under tight control. Unfortunately, the response of the pulp quality variables is extremely slow, as the dynamics of the latency chest introduces a significant lag. The long latency chest dynamics limits the maximum achievable execution frequency of any closed loop control of the pulp quality variables. Any changes made to the refining system will have an immediate impact on the quality but due to the presence of the latency chest, the changes cannot be measured immediately. The industry is in need of a rapid sampling assembly that can quickly remove latency in the pulp for analysis.

### SUMMARY OF THE INVENTION

The present invention is based in part on the development of a method and apparatus for latency removal which can be effected without introducing a significant lag time. As a result, immediate and accurate feedback is provided so that the refiner can be controlled precisely to produce mechanical pulp having the desired degree of refining. This allows the production of a maximum amount of pulp having desired properties and a minimum amount of pulp having undesired properties.

In one aspect, the invention is directed to process for analyzing a pulp material that includes the steps of:
(a) collecting a representative sample of the pulp material;
(b) adjusting the consistency of the sample, if necessary, to about 0.1 to 5% to yield an adjusted pulp sample;
(c) employing a disintegrator to release latency from the pulp that is in the adjusted pulp sample to form a pulp composition that is substantially free of latent properties; and
(d) analyzing the pulp composition to measure at least one pulp quality.

With the present invention, the pulp samples can be removed, for instance, from the blow-line of a TMP refining process for analysis. Because steps (a) through (d) can be completed in significantly less time than can be achieved with current latency removal techniques, the pulp composition data is quickly available for feedback process control. In essence, the inventive rapid sampling assembly eliminates the delay associated with the latency chest.

A method of controlling a refiner which refines cellulosic fibrous material to produce mechanical pulp having latent properties that includes the steps of:
(a) collecting a representative sample of the mechanical pulp;
(b) adjusting the consistency of the sample, if necessary, to about 0.1 to 5% to yield an adjusted pulp sample;
(c) employing a disintegrator to release latency from the pulp in that is in the adjusted pulp sample to form a pulp composition that is substantially free of latent properties;
(d) analyzing the pulp composition to measure at least one pulp quality; and
(e) in response to the analysis obtained in step (d), controlling at least one refiner parameter to insure that the mechanical pulp produced has certain desired properties.

An apparatus for analyzing a pulp material that includes:
(a) means for collecting a representative sample of the pulp material;
(b) means for adjusting the consistency of the sample to about 0.1 to 5% to yield an adjusted pulp sample;
(c) a disintegrator that is used to release latency from the pulp in that is in the adjusted sample to form a pulp composition that is substantially free of latent properties; and
(d) means for analyzing the pulp composition to measure at least one pulp quality.

### BRIEF DESCRIPTION OF THE DRAWINGS

FIG. 1 is a flow diagram of a refining line of a TMP process illustrating implementation of the rapid sampling assembly; and

FIG. 2 is a detailed depiction of the rapid sample assembly.

### DETAILED DESCRIPTION OF PREFERRED EMBODIMENTS

The present invention is directed to a pulp sampling assembly that can be employed in a continuous or batch mode. While the invention is particularly suited for use with thermo-mechanical pulp (TMP) processes, it is understood that the pulp sampling assembly can be employed with any pulping process to remove latency from pulp samples derived from various stages of the pulping process.

In a preferred embodiment of the inventive technique, a sample of high consistency pulp is diluted with hot or boiling water. After the latency is removed from the sample by hot disintegration, the sample can be analyzed with any pulp quality analyzer. Typically, the initial sample, which has a consistency of about 25% to 60%, is diluted to a consistency of about 0.1% to 5% and preferably about 4%. The term “consistency” describes the relative fiber content in a given stock quantity. Thus, an increase in consistency of the pulp stock indicates a relative increase in the dry, wood fiber constituent of a slurried, fiber-in-water suspension. The initial sample size typically ranges from about 1 to 2 liters in the batch mode or by continuous sampling. The hot disintegration step itself takes only about 1 to 5 minutes to complete so that the time required to accomplish the analysis of the pulp from obtaining the initial sampling to generating results from the pulp quality analyzer should be about 5 to 8 minutes.

In another embodiment of the invention, the signals from the pulp quality analyzer can be employed for feedback control of the refining process, e.g., TMP process, by which wood (or other fibrous raw material) is reduced to a fibrous mass which is used in papermaking. A TMP process can employ a single refiner line that empties into a latency chest or it may employ a plurality of parallel refiner lines that empty into a common latency chest. Each refiner line includes at least one refiner and preferably each line includes two or more refiners that are connected in series. Signals from the pulp quality analyzer can be applied to control any of the individual refiner lines in TMP process or any unit operation(s) thereof. Thus, the phrase the “entire” TMP process (or “entire” TMP refining process) is meant the process that is encompassed by a refiner line.

While the invention will be illustrated in connection with a single refiner line (with two refiners in the line) of a TMP process, it is understood that the present invention is applicable to any TMP process that includes at least one refiner line wherein each refiner line has at least one refiner. Moreover, while it is preferred that the refiner lines in a multiple refiner line TMP all have the same number of refiners, it is not necessary. Thus, as an example, the present invention is applicable to a TMP process that consists of a plurality of parallel refiner lines where each refiner line has a different number of refiners. Each refiner line can be controlled with the present invention.

TMP refining processes and refiner devices are known in the art and are described, for example, in U.S. Pat. No. 6,361,650 to Danielsson, et al., U.S. Pat. No. 5,016,824 to Pietinen et al., U.S. Pat. No. 4,231,842 to Ojala, U.S. Pat. No. 4,145,246 to Goheen et al., and U.S. Pat. No. 4,421,595 to Huusari, “Handbook for Pulp & Paper Technologists,” 2nd ed., G. A. Smook, 1992, Angus Wilde Publications, Inc., and Pulp and Paper Manufacture Vol III (Papermaking and Paperboard Making), R. MacDonald, ed. 1970, McGraw Hill, and Du, H., entitled, “Multivariable predictive control of a TMP plant,” Ph.D. dissertation, UBC, Vancouver, BC, Canada, 1998, which are all incorporated herein by reference.

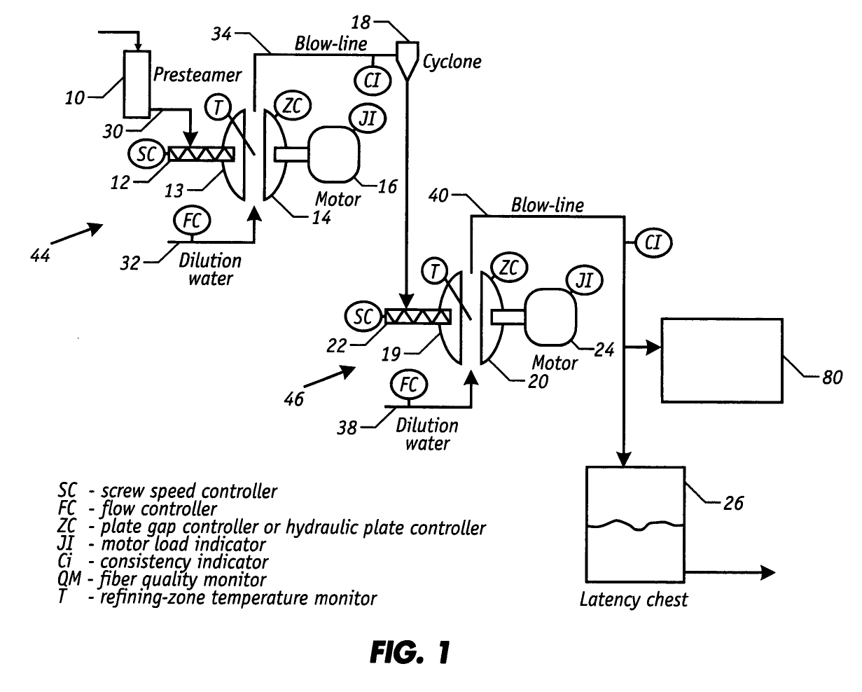

FIG. 1 illustrates a refiner line that includes a primary refiner (PR) 44 and a secondary refiner (SR) 46 that are configured in series. The refiners are preferably disc refiners. This refiner line, for instance, can be one of a plurality of parallel lines that empty into a common latency chest. The primary refiner 44 has a feed screw 12, discs 13, 14, and a motor 16. The plate gap distance is the separation of the two discs 13, 14. Water is fed to the refiner via line 38. Similarly, the secondary refiner 46 has a feed screw 22, discs 19, 22, and a motor 24. Water is supplied to the secondary refiner via line 36. Commercially available refiners such as the Sunds CD 70 refiner can be employed. A commercially available true disc clearance system can be used to regulate the plate gaps.

Raw materials, e.g., chip feed, enter the presteamer 10 of the refiner line. The presteamer 10 preferably can use both mill steam and recycled process steam to increase the chip temperature, typically to 180° C. Presteaming removes entrained air from the wood chips and induces lignin softening. The screw speed determines the volumetric feed rate to the primary refiner 44. Following the primary refiner 44 via primary blow-line 34 is a steam separator 18 that separates the semi-refined pulp and steam from the primary blow-line. The steam from the separator 18 is vented to the atmosphere or recovered in a steam recovery system.

From the separator 18, the semi-refined pulp is then fed into the secondary refiner 46 for further fiber development. At the exit of the secondary refiner 46 via secondary blow-line 40 is the latency chest 26. The latency chest 26 allows the majority of the beaten fibers to relax in hot water to remove latency. The pulp from the latency chest will be subject to a wide range of processing steps, depending on their method of preparation, and their end use. A small portion of refined pulp from the secondary blow-line 40 is diverted into the rapid sample assembly 80 for latency removal and analysis.

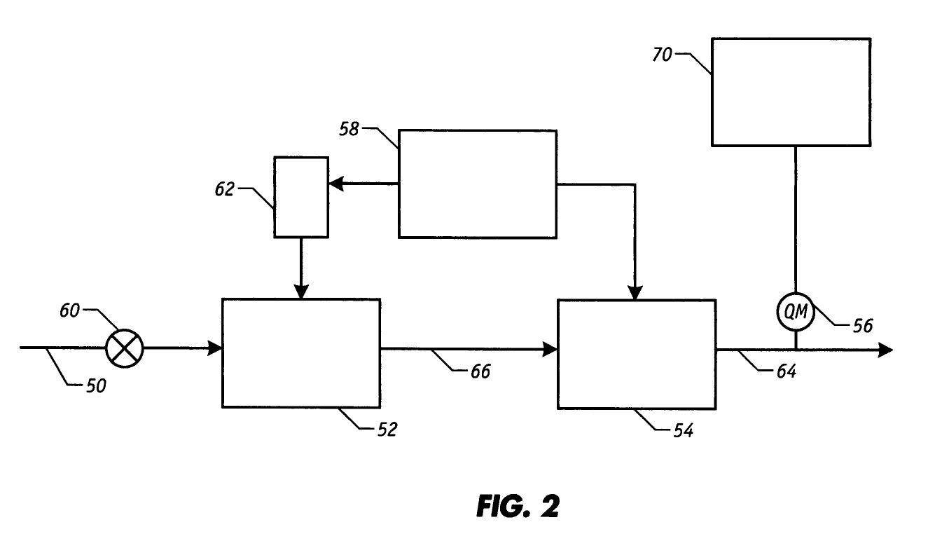

As shown in FIG. 2, by operation of valve 60, a sample of pulp is diverted from the secondary blow-line 40 via line 50 into a storage cell 52. Water from the water and cleaning fluid source 58 is added to the storage cell 52 in order to dilute the sample to a desired consistency. A heat exchanger 62 can be employed to heat the water, typically to at least about 95° C. Preferably boiling water is used to dilute the sample. After dilution is completed, the diluted sample is pumped through line 66 and into a disintegrator 54 where the desired level of latency from the pulp is quickly removed. Thereafter, as the sample exits via line 64 a standard fiber quality monitor (QM) 56 measures one or more pulp quality parameters such as Canadian standard freeness (CSF) and mean-fiber length (MFL). Fiber quality monitors are commercially available, for instance, from Metso (Finland). The pulp can be recycled to the latency chest 26 via line 64 or the pulp can be discarded.

A preferred disintegrator for removing latency employs the hot disintegration technique (also referred to as the Domtar method) by rapidly recirculating the pulp through a centrifugal pump as the pulp is maintained at a temperature of about 90° to 95° C. The energy that is imparted on the pulp removes the latency. The time to liberate latent properties can be gauged by measuring the Canadian standard freeness of the pulp, in other words, the freeness of the pulp can be been taken as an indication of release of the latent properties. The Domtar method is described, for example, in U.S. Pat. No. 4,276,119 to Karnis et al. which is incorporated herein by reference. Suitable disintegrators are commercially available from Labtech Instruments, Inc. (Laval, Canada).

The rapid sampling assembly device of the present invention can operate continuously so that successive samples of pulp can be removed from the secondary blow-line 40 for analysis. Preferably, the storage cell 52 is cleaned before a sample is placed into it. This can be accomplished by flushing the storage cell 52 with water and cleaning fluid from water and cleaning fluid source 58. The disintegrator 54 can also be similarly cleaned prior to each disintegration procedure.

Although FIG. 1 shows that the pulp is removed from the secondary blow-line 40 for analysis, it is understood that pulp can be sampled from other stages in the refining process. For example, pulp can be diverted from the primary blow-line 34 of the primary refiner 44.

As shown FIG. 1, the refiner line also includes various controllers and indicators strategically positioned along the refiner line. These instruments are all commercially available and are usually present in existing TMP mills. The true gap controller (ZC) can be substituted with a hydraulic pressure controller. An in-line blow-line consistency indicator may not be present at all mills. In the absence of an in-line sensor, software sensor based on first principle (mass & energy balances) modeling and/or empirical (multivariable statistical data analysis) modeling techniques can be used to predict blow-line consistency.

As depicted in FIG. 2, signals from the fiber quality monitor 56 are transmitted to process controller 70 which analyzes the digital signals to calculate values for various pulp parameters, e.g., CSF and MFL. In addition, the process controller includes a control system that operates in response to these measurements for controlling the operation of various components of the refiner process. The process controller 70 preferably also receives input signals from one or more other process parameter measurement devices as shown in FIG. 1, including, for example, (i) motor load indicators, (ii) consistency indicators, and (iii) refining zone temperature. In one embodiment, in response to CSF and/or MFL measurements derived from the rapid sample assembly of the present invention and preferably in conjunction with measurements from other controlled variables (CVs), the process controller 70 provides feedback control to regulate one or more manipulated variables (MVs) in order to optimize the TMP process. Table 1 lists the manipulated variables (MVs) and controlled variables (CVs) of the exemplary process. As is apparent, while these are representative of key variables, other TMP process variables can be manipulated and controlled.

TABLE 1

| MVs | CVs |
|---|---|
| Screw speed | PR motor load |
| PR dilution flow | PR blow-line consistency |
| PR plate gap | SR motor load |
| SR dilution flow | SR blow-line consistency |
| SR plate gap | Final pulp quality (MFL, CSF, Shives, |
| | Fiber length distribution) |
| Chemical | PR blow-line pulp quality (MFL, CSF, |
| addition | Shives, Fiber length distribution) |
| | SR blow-line pulp quality (MFL, CSF, |
| | Shives, Fiber length distribution) |
| | PR specific energy |
| | SR specific energy |
| Steam flow | Total specific energy (PR + SR) |
| | Power split ratio between PR and SR |
| | Refining Zone temperature |

TMP controller design can employ single-loop Proportional-integral-derivative (PID) based decentralized control architecture. PID based control strategy is acceptable for regulation of local control loops such as flow and pressure regulation, but a PID controller cannot handle complex multivariable dynamics. In addition, a PID controller can only control a single process output. However, to adequately control pulp quality at least two variables such as, for example, CSF and MFL must be controlled. Since these variables are physically linked, they cannot be independently controlled to arbitrary targets, instead these variables must be controlled within an operator defined quality window. The quality window is defined by setting the upper and lower limits on the pulp quality variables. In order to handle this control problem a multivariable controller is required that can also handle process constraints. Constrained model based predictive control (MPC) is a natural candidate in the process industry. MPC provides a unified framework to efficiently handle complex process interactions and constraints. MPC technology has also gained industrial acceptance and it can be easily integrated into existing mill distributed control system platforms.

U.S. Patent Application Publication 2005/0263259 entitled “System and Method for Controlling a Thermo-Mechanical Wood Pulp Refiner,” to Sidhu et al. and assigned to Honeywell International, Inc., which is incorporated herein by reference, describes a technique of extending decentralized control architecture and strategy extended into a centralized controller design framework. However, this strategy cannot be directly extended to a centralized framework by utilizing a single MPC controller. Since the process dynamics are spread over a wide frequency range, a single MPC, executing at a fixed frequency, would not be able to provide adequate control of both fast and slow dynamics. In order to mitigate this problem, a two-level control strategy was developed.

Specifically, method disclosed in the '336 patent application applied MPC technology in a two-level control strategy that can control the entire TMP refining line to increase throughput, reduce energy usage and improve pulp quality. The control strategy leverages the natural decoupling in the process dynamics. As a result, Model Predicative Range Control controllers can be designed to independently regulate the fast and slow dynamics of the process. The first level is the Stabilization Controller that preferably regulates the refiner line motor loads and the blow-line consistencies. The second level is the Quality Controller that preferably controls the slow dynamics associated with the pulp quality variables. The Quality Controller can directly manipulate the plate gap to control the final pulp quality. The direct manipulation of the plate gap removes the requirement to implement an internal specific energy loop. However a specific energy loop on each refiner can be included. In this control strategy the designer can independently select the execution frequency of the two levels. By operating the refiner lines at the maximum allowable motor loads the production is automatically maximized for a given pulp quality window. This modular approach can also handle multiple refiner lines that empty into a common latency chest. In order to integrate and coordinate the Stabilization and Quality Controllers, an Optimizer based on distributed quadratic programming was also developed. The Optimizer performs a global optimization of the process and improves the overall constraint handling of the control strategy. With the use of the inventive sampling assembly, the quality control layer can be directly incorporated into the stabilization layer since fast pulp quality measurements will be available and the issue of fast and slow dynamics is no longer a problem. The coordination of multiple refiner lines can still be accomplished using the Optimizer. This approach would be a subset of the methodology described in U.S. Patent Application Publication No. 2005/0263259.

The foregoing has described the principles, preferred embodiments and modes of operation of the present invention. However, the invention should not be construed as being limited to the particular embodiments discussed. Thus, the above-described embodiments should be regarded as illustrative rather than restrictive, and it should be appreciated that variations may be made in those embodiments by workers skilled in the art without departing from the scope of the present invention as defined by the following claims.

What is claimed is:

1. A process for analyzing a pulp material that comprises the steps of:
(a) collecting a representative sample of the pulp material;
(b) adjusting the consistency of the sample, if necessary, to about 0.1 to 5% to yield an adjusted pulp sample;
(c) employing a disintegrator to release latency from the pulp that is in the adjusted pulp sample to form a pulp composition that is substantially free of latent properties; and
(d) analyzing the pulp composition to measure at least one pulp quality.

2. The process of claim 1 wherein step (b) comprises diluting the sample with water that is at a temperature of at least 95° C.

3. The process of claim 2 wherein the sample of pulp material in step (a) has a consistency of about 4% and step (b) comprises diluting the sample with boiling water.

4. The process of claim 1 wherein step (c) comprises re-circulating the adjusted pulp sample until a desired level of latency is removed.

5. The process of claim 1 wherein step (d) comprises measuring at least one pulp variable.

6. The process of claim 1 wherein the process does not require a latency chest.

7. A process of controlling a refiner which refines cellulosic fibrous material to produce mechanical pulp having latent properties that comprises the steps of:
(a) collecting a representative sample of the mechanical pulp;
(b) adjusting the consistency of the sample, if necessary, to about 0.1 to 5% to yield an adjusted pulp sample;
(c) employing a disintegrator to release latency from the pulp that is in the adjusted pulp sample to form a pulp composition that is substantially free of latent properties;
(d) analyzing the pulp composition to measure at least one pulp quality; and
(e) in response to the analysis obtained in step (d), controlling at least one refiner parameter to insure that the mechanical pulp produced has certain desired properties.

8. The process of claim 7 wherein step (b) comprises diluting the sample with water that is at a temperature of at least 95° C.

9. The process of claim 8 wherein the sample of pulp material in step (a) has a consistency of about 4% and step (b) comprises diluting the sample with boiling water.

10. The process of claim 7 wherein step (c) comprises re-circulating the adjusted pulp sample until a desired level of latency is removed.

11. The process of claim 7 wherein step (d) comprises measuring at least one pulp variable.

12. The process of claim 7 wherein the process does not require a latency chest.

13. The process of claim 7 wherein in step (a) the sample of mechanical pulp is derived from a mechanical pulp refining process.

14. The process of claim 13 wherein the at least one refiner parameter is a manipulated variable that is subject to automatic manipulation.

15. The process of claim 14 wherein the manipulated variable is selected from the group consisting of (i) dilution flow into a refiner, (ii) plate gap distance between the refiner discs, (iii) rate of chemical addition, and (iv) steam flow into the cellulosic fibrous material.

16. The process of claim 7 wherein step (a) comprises collecting sample from a blow-line of a refiner.

17. An apparatus for analyzing a pulp material that comprises:
(a) means for collecting a representative sample of the pulp material;
(b) means for adjusting the consistency of the sample to about 0.1 to 5% to yield an adjusted pulp sample;
(c) a disintegrator that is used to release latency from the pulp that is in the adjusted sample to form a pulp composition that is substantially free of latent properties; and
(d) means for analyzing the pulp composition to measure at least one pulp quality.

18. The apparatus of claim 17 wherein the means for collecting the representative sample removes a sample from a blow line of a thermo-mechanical pulp refiner that produces mechanical pulp.

19. The apparatus of claim 18 further comprising (e) means for generating a signal to control at least one refiner parameter to insure that the mechanical pulp produced has certain desired properties.

20. The apparatus of claim 17 with the proviso that the apparatus does not use a latency chest.
